(19) United States
(12) Patent Application Publication
(10) Pub. No.: US 2023/0405007 A1
(43) Pub. Date: Dec. 21, 2023

(54) COMPOUND FOR USE IN AND METHODS OF TREATMENT OF FIBROTIC DISEASES

(71) Applicant: Galapagos NV, Mechelen (BE)

(72) Inventor: Silke Wilma HUETTNER, Mechelen (BE)

(21) Appl. No.: 18/038,667

(22) PCT Filed: Nov. 22, 2021
(86) PCT No.: PCT/EP2021/082532

§ 371 Date: May 24, 2023

(30) Foreign Application Priority Data
Nov. 24, 2020 (EP) 20209647.5

(51) Int. Cl. A61K 31/519; A61K 31/4418; A61P 11/00

(52) U.S. Cl. CPC A61K 31/519; A61K 31/4418; A61P 11/00

(57) ABSTRACT

The present invention relates to improved methods for the use of a compound according to formula I in the treatment of fibrosis, in particular idiopathic pulmonary fibrosis (IPF), by contraindicating combined therapy with specific IPF therapeutic compounds, where the combined therapy results in a poorer adverse event profile.

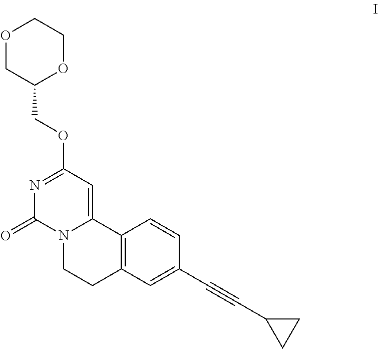 [SMILES: O=c1nc(OC[C@@H]2COCCO2)cc2n1CCc1cc(C#CC3CC3)ccc1-2]

### FIELD OF THE INVENTION

The present invention relates to improved methods for the use of Compound 1 in the treatment of fibrosis, in particular idiopathic pulmonary fibrosis (IPF), by contraindicating combined therapy with specific IPF therapeutic compounds, where the combined therapy results in a poorer adverse event profile.

### BACKGROUND OF THE INVENTION

Fibrosis is a process that can be triggered by chronic tissue damage because of toxic substances, viral infection, inflammation, or mechanical stress (Nanthakumar et al., 2015); and may be defined as the abnormal or excessive production and accumulation of extracellular matrix (ECM).

In particular, fibrosis is a key driver of progressive organ dysfunction in many inflammatory and metabolic diseases, including idiopathic pulmonary fibrosis (IPF). These conditions remain poorly treated despite advances in the understanding of the disease mechanism and, more recently, an increase in the number of clinical trials reflecting the need to identify new treatments, particularly in IPF (Nanthakumar et al., 2015).

Compound 1 is a small molecule drug under investigation for the treatment of IPF, the structure, name and synthesis are known and described in WO 2013/092791 and WO2019/096944. Compound 1 is being developed as a GPR84 antagonist and is being studied for the treatment of pulmonary fibrosis, and idiopathic pulmonary fibrosis in particular (“A Clinical Study to Test How Effective and Safe GLPG1205 is for Patients with Idiopathic Pulmonary Fibrosis (IPF) (PINTA)”, Clinical Trial: NCT03725852 and “Characterization of GLPG1205 in Mouse Fibrosis Models: A Potent and Selective Antagonist of GPR84 for Treatment of Idiopathic Pulmonary Fibrosis” American Journal of Respiratory and Critical Care Medicine, 2020; 201: A1046).

GPR84 (also known as EX33) has been isolated and characterized from human B cells (Wittenberger et al., 2001) and also using a degenerate primer reverse transcriptase-polymerase chain reaction (RT-PCR) approach (Yousefi et al., 2001). It remained an orphan GPCR until the identification of medium-chain Free Fatty Acids (FFAs) with carbon chain lengths of 9-14 as ligands for this receptor (Wang et al., 2006).

GPR84 is activated by medium-chain FFAs, such as capric acid (C10:0), undecanoic acid (C11:0) and lauric acid (12:0) which amplify lipopolysaccharide stimulated production of pro-inflammatory cytokines/chemokines (TNFa, IL-6, IL-8, CCL2 and others), and is highly expressed in neutrophils and monocytes (macrophages). (Miyamoto et al., 2016). In contrast, GPR84-ligand mediated chemotaxis of neutrophils and monocytes/macrophages is inhibited by GPR84 antagonists. (Suzuki et al., 2013)

Nintedanib is one of the currently approved treatments for idiopathic pulmonary fibrosis under the brand name Ofev® (https://www.ema.europa.eu/en/medicines/human/EPAR/ofev).

Despite the approval of nintedanib (Ofev®) and pirfenidone (Esbriet®), for the treatment of IPF, unfortunately there remains a high unmet medical need in this disease. Due to the complexity of the pathology of IPF and the activation of multiple coactivated pathways, it is expected that combination therapies will play an essential role in the treatment of IPF (Wuyts et al., Lancet Respir Med 2014, 2: 933-42), therefore it is crucial to understand not only which combinations result in enhanced and synergistic efficacy, but also which combinations must be contraindicated.

The present invention relates to Compound 1 for use in the treatment of IPF, wherein treatment of the patient with nintedanib must be discontinued either prior to, or simultaneously with, the administration of Compound 1.

### SUMMARY OF THE INVENTION

The invention described herein is based upon the observation of increased serious adverse events (SAEs) and/or treatment emergent adverse events (TEAEs) in therapy wherein the Compound of the Invention was administered in combination with or alongside nintedanib.

Accordingly, in a first aspect the present invention provides the Compound of the Invention for use in treating a patient in need of the Compound of the Invention therapy, characterized in that the treating comprises avoiding, contraindicating or discontinuing concomitant use or co-administration of nintedanib.

In a further aspect, the present invention also provides the use of the Compound of the Invention in the manufacture of a medicament for treating a patient in need of therapy using the Compound of the Invention, characterized in that the treating comprises avoiding, contraindicating or discontinuing concomitant use or co-administration of nintedanib.

In a another aspect, the present invention provides a method of administering treatment using the Compound of the Invention to a patient in need of therapy using the Compound of the Invention comprising administering the patient a therapeutically effective amount of the Compound of the Invention, and, avoiding, contraindicating or discontinuing concomitant use or co-administration of nintedanib.

In specific embodiments the patient in need of therapy is a patient suffering from fibrotic diseases, such as pulmonary fibrosis, in particular idiopathic pulmonary fibrosis (IPF) and progressive fibrosing interstitial lung disease (PF-ILD). Most particularly, the patient in need of therapy suffers from IPF.

Compound 1 means a compound according to formula I below:

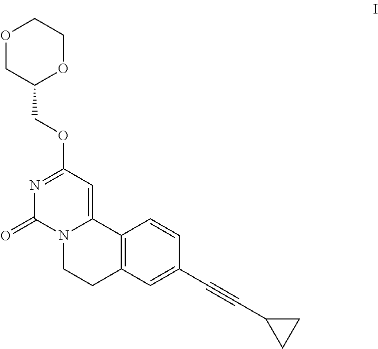 [SMILES: O=c1nc(OC[C@@H]2COCCO2)cc2n1CCc1cc(C#CC3CC3)ccc1-2]

The chemical name of Compound 1 is “9-Cyclopropylethynyl-2-((S)-1-[1,4]dioxan-2-ylmethoxy)-6,7-dihydro-pyrimido[6,1-a]iso quinolin-4-one”.

Moreover, Compound 1, useful in the pharmaceutical compositions and treatment methods disclosed herein, is pharmaceutically acceptable as prepared and used.

Other objects and advantages will become apparent to those skilled in the art from a consideration of the ensuing detailed description.

It will be appreciated that the compound of the invention may be metabolized to yield biologically active metabolites.

### DETAILED DESCRIPTION OF THE INVENTION

### Definitions

The following terms are intended to have the meanings presented therewith below and are useful in understanding the description and intended scope of the present invention.

When describing the invention, which may include the compound of formula I, pharmaceutical compositions containing said compound and methods of using said compound and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein.

The articles ‘a’ and ‘an’ may be used herein to refer to one or to more than one (i.e. at least one) of the grammatical objects of the article. By way of example ‘an analogue’ means one analogue or more than one analogue.

“The Compound of the Invention” means the compound of formula I or Compound 1, which expression includes the pharmaceutically acceptable salts, and the solvates, e.g. hydrates, and the solvates of the pharmaceutically acceptable salts where the context so permits. Similarly, reference to intermediates, whether or not they themselves are claimed, is meant to embrace their salts, and solvates, where the context so permits.

‘Pharmaceutically acceptable’ means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

‘Pharmaceutically acceptable salt’ refers to a salt of Compound 1 that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g. an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. The term ‘pharmaceutically acceptable cation’ refers to an acceptable cationic counter-ion of an acidic functional group. Such cations are exemplified by sodium, potassium, calcium, magnesium, ammonium, tetraalkylammonium cations, and the like.

‘Pharmaceutically acceptable vehicle’ refers to a diluent, adjuvant, excipient or carrier with which the compound of the invention is administered.

‘Solvate’ refers to forms of the compound that are associated with a solvent, usually by a solvolysis reaction. This physical association includes hydrogen bonding. Conventional solvents include water, EtOH, acetic acid and the like. The compound of the invention may be prepared e.g. in crystalline form and may be solvated or hydrated. Suitable solvates include pharmaceutically acceptable solvates, such as hydrates, and further include both stoichiometric solvates and non-stoichiometric solvates. In certain instances the solvate will be capable of isolation, for example when one or more solvent molecules are incorporated in the crystal lattice of the crystalline solid. ‘Solvate’ encompasses both solution-phase and isolable solvates. Representative solvates include hydrates, ethanolates and methanolates.

‘Subject’ includes humans. The terms ‘human’, ‘patient’ and ‘subject’ are used interchangeably herein.

‘Effective amount’ means the amount of the Compound of the Invention that, when administered to a subject for treating a disease, is sufficient to effect such treatment for the disease. The “effective amount” can vary depending on the disease and its severity, and the age, weight, etc., of the subject to be treated.

‘Preventing’ or ‘prevention’ refers to a reduction in risk of acquiring or developing a disease or disorder (i.e. causing at least one of the clinical symptoms of the disease not to develop in a subject that may be exposed to a disease-causing agent, or predisposed to the disease in advance of disease onset.

The term ‘prophylaxis’ is related to ‘prevention’, and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

‘Treating’ or ‘treatment’ of any disease or disorder refers, in one embodiment, to ameliorating the disease or disorder (i.e. arresting the disease or reducing the manifestation, extent or severity of at least one of the clinical symptoms thereof). In another embodiment ‘treating’ or ‘treatment’ refers to ameliorating at least one physical parameter, which may not be discernible by the subject. In yet another embodiment, ‘treating’ or ‘treatment’ refers to modulating the disease or disorder, either physically, (e.g. stabilization of a discernible symptom), physiologically, (e.g. stabilization of a physical parameter), or both. In a further embodiment, “treating” or “treatment” relates to slowing the progression of the disease.

As used herein the term ‘fibrotic diseases’ refers to diseases characterized by excessive scarring due to excessive production, deposition, and contraction of extracellular matrix, and are that are associated with the abnormal accumulation of cells and/or fibronectin and/or collagen and/or increased fibroblast recruitment and include but are not limited to fibrosis of individual organs or tissues such as the heart, kidney, liver, joints, lung, pleural tissue, peritoneal tissue, skin, cornea, retina, musculoskeletal and digestive tract. In particular, the term fibrotic diseases refers to idiopathic pulmonary fibrosis (IPF); cystic fibrosis, other diffuse parenchymal lung diseases of different etiologies including iatrogenic drug-induced fibrosis, occupational and/or environmental induced fibrosis, granulomatous diseases (sarcoidosis, hypersensitivity pneumonia), collagen vascular disease, alveolar proteinosis, Langerhans cell granulomatosis, lymphangioleiomyomatosis, inherited diseases (Hermansky-Pudlak Syndrome, tuberous sclerosis, neurofibromatosis, metabolic storage diseases, familial interstitial lung disease); radiation induced fibrosis; chronic obstructive pulmonary disease; scleroderma; bleomycin induced pulmonary fibrosis; chronic asthma; silicosis; asbestos induced pulmonary fibrosis; acute respiratory distress syndrome (ARDS); kidney fibrosis; tubulointerstitium fibrosis; glomerular nephritis; diabetic nephropathy, focal segmental glomerular sclerosis; IgA nephropathy; hypertension; Alport; gut fibrosis; liver fibrosis; cirrhosis; alcohol induced liver fibrosis; toxic/drug induced liver fibrosis; hemochromatosis; alcoholic steato hepatitis (ASH), nonalcoholic steatohepatitis (NASH), nonalcoholic fatty liver disease (NAFLD); cholestasis, biliary duct injury; primary sclerosing cholangitis (PSC), primary biliary cirrhosis (PBC); infection induced liver fibrosis; viral induced liver fibrosis; and autoimmune hepatitis; corneal scarring; hypertrophic scarring; Dupuytren disease, keloids, cutaneous fibrosis; cutaneous scleroderma; systemic sclerosis, spinal cord injury/fibrosis; myelofibrosis; Duchenne muscular dystrophy (DMD) associated musculoskeletal fibrosis, vascular restenosis; atherosclerosis; arteriosclerosis; Wegener's granulomatosis; Peyronie's disease, or chronic lymphocytic. More particularly, the term “fibrotic diseases” refers to idiopathic pulmonary fibrosis (IPF), Dupuytren disease, nonalcoholic steatohepatitis (NASH), nonalcoholic fatty liver disease (NAFLD), Alcoholic steato hepatitis, (ASH), portal hypertension, systemic sclerosis, renal fibrosis, and cutaneous fibrosis. Most particularly, the term “fibrotic diseases” refers to nonalcoholic steatohepatitis (NASH), and/or nonalcoholic fatty liver disease (NAFLD). In a specific aspect, the term fibrotic diseases refers to pulmonary fibrosis, specifically in particular idiopathic pulmonary fibrosis (IPF) and progressive fibrosing interstitial lung disease (PF-ILD). Alternatively, most particularly, the term “fibrotic diseases” refers to IPF.

As used herein the term “Adverse Event (AE)” refers to any untoward medical occurrence in a patient or clinical investigation subject administered a pharmaceutical product and which does not necessarily have to have a causal relationship with this treatment. An Adverse Event (AE) can therefore be any unfavourable and/or unintended sign (including an abnormal laboratory finding, for example), symptom, or disease temporally associated with the use of a medicinal product, whether or not considered related to the medicinal product. AEs may also include pre- or post-treatment complications that occur as a result of protocol mandated procedures, lack of efficacy, Overdose or drug Abuse/Misuse reports. Pre-existing events that increase in severity or change in nature during or as a consequence of participation in the Clinical Study will also be considered AEs.

As used herein the term “Treatment Emergent Adverse Event (TEAE)” refers to any Adverse Event (AE) (or worsening of any Adverse Event (AE)) with an onset date on or after the start date of the respective treatment and no later than 30 days after the last dose of the respective treatment.

As used herein the term “Serious Adverse Event (SAEs)” refers to an Adverse Event (AE) that results in one of the following: death, a life-threatening event (an event in which the subject was at risk of death at the time of the event; it does not refer to an event that hypothetically might have caused death if it were more severe), in-subject hospitalization or prolongation of existing hospitalization, persistent or significant disability/incapacity, a congenital anomaly/birth defect, or a medically significant event (medical and scientific judgment should be exercised in deciding whether other situations should be considered serious such as important medical events that might not be immediately life-threatening or result in death or hospitalization but might jeopardize the subject or might require intervention to prevent one of the other outcomes listed in the definition above).

When ranges are referred to herein, for example but without limitation, C₁₋₈ alkyl, the citation of a range should be considered a representation of each member of said range.

The present disclosure includes all isotopic forms of the Compound of the Invention provided herein, whether in a form (i) wherein all atoms of a given atomic number have a mass number (or mixture of mass numbers) which predominates in nature (referred to herein as the “natural isotopic form”) or (ii) wherein one or more atoms are replaced by atoms having the same atomic number, but a mass number different from the mass number of atoms which predominates in nature (referred to herein as an “unnatural variant isotopic form”). It is understood that an atom may naturally exists as a mixture of mass numbers. The term “unnatural variant isotopic form” also includes embodiments in which the proportion of an atom of given atomic number having a mass number found less commonly in nature (referred to herein as an “uncommon isotope”) has been increased relative to that which is naturally occurring e.g. to the level of >20%, >50%, >75%, >90%, >95% or >99% by number of the atoms of that atomic number (the latter embodiment referred to as an “isotopically enriched variant form”). The term “unnatural variant isotopic form” also includes embodiments in which the proportion of an uncommon isotope has been reduced relative to that which is naturally occurring. Isotopic forms may include radioactive forms (i.e. they incorporate radioisotopes) and non-radioactive forms. Radioactive forms will typically be isotopically enriched variant forms.

An unnatural variant isotopic form of a compound may thus contain one or more artificial or uncommon isotopes such as deuterium (²H or D), carbon-11 (¹¹C), carbon-13 (¹³C), carbon-14 (¹⁴C), nitrogen-13 (¹³N), nitrogen-15 (¹⁵N), oxygen-15 (¹⁵O), oxygen-17 (¹⁷O), oxygen-18 (¹⁸O), phosphorus-32 (³²P), sulphur-35 (³⁵S), chlorine-36 (³⁶Cl), chlorine-37 (³⁷Cl), fluorine-18 (¹⁸F) iodine-123 (¹²³I), iodine-125 (¹²⁵I) in one or more atoms or may contain an increased proportion of said isotopes as compared with the proportion that predominates in nature in one or more atoms.

Unnatural variant isotopic forms comprising radioisotopes may, for example, be used for drug and/or substrate tissue distribution studies. The radioactive isotopes tritium, i.e. ³H, and carbon-14, i.e. ¹⁴C, are particularly useful for this purpose in view of their ease of incorporation and ready means of detection. Unnatural variant isotopic forms which incorporate deuterium i.e ²H or D may afford certain therapeutic advantages resulting from greater metabolic stability, for example, increased in vivo half-life or reduced dosage requirements, and hence may be preferred in some circumstances. Further, unnatural variant isotopic forms may be prepared which incorporate positron emitting isotopes, such as ¹¹C, ¹⁸F, ¹⁵O and ¹³N, and would be useful in Positron Emission Topography (PET) studies for examining substrate receptor occupancy.

It is also to be understood that compounds that have the same molecular formula but differ in the nature or sequence of bonding of their atoms or the arrangement of their atoms in space are termed ‘isomers’. Isomers that differ in the arrangement of their atoms in space are termed ‘stereoisomers’.

Stereoisomers that are not mirror images of one another are termed ‘diastereomers’ and those that are non-superimposable mirror images of each other are termed ‘enantiomers’. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e. as (+) or (−)-isomers respectively). A chiral compound can exist as either individual enantiomer or as a mixture thereof. A mixture containing equal proportions of the enantiomers is called a ‘racemic mixture’.

‘Tautomers’ refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane that are likewise formed by treatment with acid or base.

Tautomeric forms may be relevant to the attainment of the optimal chemical reactivity and biological activity of a compound of interest.

It will be appreciated that the Compound of the Invention may be metabolized to yield biologically active metabolites.

### Methods of Treatment

In one embodiment, the present invention provides the Compound of the Invention, or pharmaceutical compositions comprising the Compound of the Invention, for use in the treatment of a patient in need of therapy using the Compound of the Invention, characterized in that the treating comprises avoiding, contraindicating or discontinuing concomitant use or co-administration of nintedanib. In a particular embodiment, the patient in need of therapy is suffering from one or more fibrotic diseases. In a particular embodiment, the fibrotic disease is pulmonary fibrosis. In a most particular embodiment, the pulmonary fibrosis is PF-ILD. In another most particular embodiment, the fibrotic disease is idiopathic pulmonary fibrosis (IPF).

In another embodiment, the present invention provides the Compound of the Invention, or pharmaceutical compositions comprising the Compound of the Invention for use in the manufacture of a medicament for use in the treatment of a patient in need of therapy using the Compound of the Invention, characterized in that the treatment comprises avoiding, contraindicating or discontinuing concomitant use or co-administration of nintedanib. In a particular embodiment, the patient in need of therapy suffers from one or more fibrotic diseases. In a particular embodiment, the fibrotic disease is pulmonary fibrosis. In a most particular embodiment, the pulmonary fibrosis is PF-ILD. In another most particular embodiment, the fibrotic disease is idiopathic pulmonary fibrosis (IPF).

In additional method of treatment aspects, this invention provides methods of treatment of a patient in need thereof, which methods comprise the administration of an effective amount of the Compound of the Invention or one or more of the pharmaceutical compositions herein described wherein the treatment additionally comprises avoiding, contraindicating or discontinuing concomitant use or co-administration of nintedanib. In a particular embodiment, the patient in need of therapy suffers from one or more fibrotic diseases. In a particular embodiment, the fibrotic disease is pulmonary fibrosis. In a most particular embodiment, the pulmonary fibrosis is PF-ILD. In another most particular embodiment, the fibrotic disease is idiopathic pulmonary fibrosis (IPF).

Injection dose levels range from about 0.1 mg/kg/h to at least 10 mg/kg/h, all for from about 1 to about 120 h and especially 24 to 96 h. A preloading bolus of from about 0.1 mg/kg to about 10 mg/kg or more may also be administered to achieve adequate steady state levels. The maximum total dose is not expected to exceed about 1 g/day for a 40 to 80 kg human patient.

For the prophylaxis and/or treatment of long-term conditions, such as degenerative conditions, the regimen for treatment usually stretches over many months or years so oral dosing is preferred for patient convenience and tolerance. With oral dosing, one to four (1-4) regular doses daily, especially one to three (1-3) regular doses daily, typically one to two (1-2) regular doses daily, and most typically one (1) regular dose daily are representative regimens. Alternatively, for long lasting effect drugs, with oral dosing, once every other week, once weekly, and once a day are representative regimens. In particular, dosage regimen can be every 1-14 days, more particularly 1-10 days, even more particularly 1-7 days, and most particularly 1-3 days.

Using these dosing patterns, each dose provides from about 1 to about 1000 mg of the Compound of the Invention, with particular doses each providing from about 10 to about 500 mg and especially about to about 250 mg. In a particular embodiment, the Compound of the Invention is administered in a 30 to 250 mg (such as 50 or 100 mg) daily dose for the treatment and/or prevention of fibrotic disease, more in particular the treatment and/or prevention IPF. In a specific embodiment the Compound of the Invention is administered in fixed dosage units of 50 mg for a total dose of 50 mg per day or 100 mg per day.

Transdermal doses are generally selected to provide similar or lower blood levels than are achieved using injection doses.

According to the methods of the present invention, the Compound of the Invention can be administered as the sole active agent or it can be administered in combination with other therapeutic agents, provided that concomitant use or co-administration treatment with nintedanib is avoided, contraindicated or discontinued, said other therapeutic agents may demonstrate the same or a similar therapeutic activity and that are determined to be safe and efficacious for such combined administration. In a specific embodiment, co-administration of two (or more) agents allows for significantly lower doses of each to be used, thereby reducing the side effects seen.

In one embodiment, the compound of the invention or a pharmaceutical composition comprising the compound of the invention is administered as a medicament. In a specific embodiment, said pharmaceutical composition additionally comprises a further active ingredient, provided said further active ingredient is not nintedanib.

In one embodiment the Compound of the Invention is not an isotopic variant.

In one aspect the Compound of the Invention according to any one of the embodiments herein described is present as the free base.

In one aspect the Compound of the Invention according to any one of the embodiments herein described is a pharmaceutically acceptable salt.

In one aspect the Compound of the Invention according to any one of the embodiments herein described is a solvate of the compound.

In one aspect the Compound of the Invention according to any one of the embodiments herein described is a solvate of a pharmaceutically acceptable salt of the Compound of the Invention.

Alternatively, the exclusion of one or more of the specified variables from a group or an embodiment, or combinations thereof is also contemplated by the present invention.

### Pharmaceutical Compositions

When employed as a pharmaceutical, the Compound of the Invention is typically administered in the form of a pharmaceutical composition. Such compositions can be prepared in a manner well known in the pharmaceutical art and comprise at least one active compound of the invention according to Formula I. Generally, the Compound of the Invention is administered in a pharmaceutically effective amount. The amount of compound of the invention actually administered will typically be determined by a physician, in the light of the relevant circumstances, including the condition to be treated, the chosen route of administration, the actual compound of the invention administered, the age, weight, and response of the individual patient, the severity of the patient's symptoms, and the like.

The pharmaceutical compositions of this invention can be administered by a variety of routes including oral, rectal, transdermal, subcutaneous, intra-articular, intravenous, intramuscular, and intranasal. Depending on the intended route of delivery, the Compound of the Invention is preferably formulated as either injectable or oral compositions or as salves, as lotions or as patches all for transdermal administration.

The compositions for oral administration can take the form of bulk liquid solutions or suspensions, or bulk powders. More commonly, however, the compositions are presented in unit dosage forms to facilitate accurate dosing. The term ‘unit dosage forms’ refers to physically discrete units suitable as unitary dosages for human subjects and other mammals, each unit containing a predetermined quantity of active material calculated to produce the desired therapeutic effect, in association with a suitable pharmaceutical excipient, vehicle or carrier. Typical unit dosage forms include prefilled, premeasured ampules or syringes of the liquid compositions or pills, tablets, capsules or the like in the case of solid compositions. In such compositions, the compound of the invention according to Formula I is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or non-aqueous vehicle with buffers, suspending and dispensing agents, colorants, flavors and the like. Solid forms may include, for example, any of the following ingredients, or compound of the inventions of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint or orange flavoring.

Injectable compositions are typically based upon injectable sterile saline or phosphate-buffered saline or other injectable carriers known in the art. As before, the active compound of the invention according to Formula I in such compositions is typically a minor component, often being from about 0.05 to 10% by weight with the remainder being the injectable carrier and the like.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10% by weight, and more preferably from about 0.5 to about 15% by weight. When formulated as an ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope of this invention.

The Compound of the Invention can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17^{th} edition, 1985, Mack Publishing Company, Easton, Pennsylvania, which is incorporated herein by reference.

The Compound of the Invention can also be administered in sustained release forms or from sustained release drug delivery systems. A description of representative sustained release materials can be found in Remington's Pharmaceutical Sciences.

The following formulation examples illustrate representative pharmaceutical compositions that may be prepared in accordance with this invention. The present invention, however, is not limited to the following pharmaceutical compositions.

### Formulation 1—Tablets

The Compound of the Invention according to Formula I may be admixed as a dry powder with a dry gelatin binder in an approximate 1:2 weight ratio. A minor amount of magnesium stearate may be added as a lubricant. The mixture may be formed into 240-270 mg tablets (80-90 mg of active compound of the invention according to Formula I per tablet) in a tablet press.

### Formulation 2—Capsules

The Compound of the Invention according to Formula I may be admixed as a dry powder with a starch diluent in an approximate 1:1 weight ratio. The mixture may be filled into 250 mg capsules (125 mg of active compound of the invention according to Formula I per capsule).

### Formulation 3—Liquid

The Compound of the Invention according to Formula I (125 mg), may be admixed with sucrose (1.75 g) and xanthan gum (4 mg) and the resultant mixture may be blended, passed through a No. 10 mesh U.S. sieve, and then mixed with a previously made solution of microcrystalline cellulose and sodium carboxymethyl cellulose (11:89, 50 mg) in water. Sodium benzoate (10 mg), flavor, and color may be diluted with water and added with stirring. Sufficient water may then be added with stirring. Further sufficient water may be then added to produce a total volume of 5 mL.

### Formulation 4—Tablets

The Compound of the Invention according to Formula I may be admixed as a dry powder with a dry gelatin binder in an approximate 1:2 weight ratio. A minor amount of magnesium stearate may be added as a lubricant. The mixture may be formed into 450-900 mg tablets (150-300 mg of active compound of the invention according to Formula I) in a tablet press.

### Formulation 5—Injection

The Compound of the Invention according to Formula I may be dissolved or suspended in a buffered sterile saline injectable aqueous medium to a concentration of approximately 5 mg/mL.

### Formulation 6—Topical

Stearyl alcohol (250 g) and a white petrolatum (250 g) may be melted at about 75° C. and then a mixture of the Compound of the Invention according to Formula I (50 g) methylparaben (0.25 g), propylparaben (0.15 g), sodium lauryl sulfate (10 g), and propylene glycol (120 g) dissolved in water (about 370 g) may be added and the resulting mixture may be stirred until it congeals.

### BIOLOGICAL EXAMPLES

The methods for the preparation of the Compound of the Invention and biological examples have been described in, inter alia, WO 2013/092791 and WO 2019/096944.

### CLINICAL EXAMPLES

TABLE I
List of abbreviations used herein:

| Abbreviation | Definition |
|---|---|
| 6MWT | 6-Minute Walk Test |
| AE | adverse event |
| ALAT | Latin American Thoracic Association |
| ALT | alanine aminotransferase |
| AST | aspartate aminotransferase |
| ATS | American Thoracic Society |
| DLCO | diffusing capacity for the lungs for carbon monoxide |
| DNA | deoxyribonucleic acid |
| ECG | electrocardiogram |
| ERS | European Respiratory Society |
| FEV1 | forced expiratory volume in one second |
| FRI | functional respiratory imaging |
| FVC | forced vital capacity |
| HRCT | high-resolution computed tomography |
| IMP | investigational medicinal product |
| IPF | idiopathic pulmonary fibrosis |
| JRS | Japanese Respiratory Society |
| SGRQ | St. George's Respiratory Questionnaire |

### Example 1. Idiopathic Pulmonary Fibrosis (IPF) Clinical Study

The study of the current example was a randomized, double-blind, parallel group, placebo-controlled, multicenter, Phase II study to evaluate the efficacy, safety and tolerability of Compound 1 in subjects with idiopathic pulmonary fibrosis (IPF). The primary objective of this study was to evaluate the efficacy of Compound 1 treatment in subjects with IPF on pulmonary function as evaluated by FVC compared to placebo over 26 weeks.

### 1.1. Study Endpoints

Primary outcome measure was:
Change from baseline in FVC (mL) over 26 weeks compared to placebo.

Secondary outcome measures were:
Safety and tolerability changes over time (baseline to 26 weeks).
Time to any of major events (whichever occurs first) defined as:
   Respiratory-related death
   First hospitalization (all-cause and respiratory-related)
   Need to be placed on a lung transplant list during the study
Change from baseline 26 weeks in functional exercise capacity, assessed by the 6MWT at Week 26.
Change from baseline until 26 weeks in quality of life measures, assessed by the St. George's Respiratory Questionnaire (SGRQ) total score and domains and proportion of SGRQ responders.
Concentrations of Compound 1, nintedanib and pirfenidone.

Other outcome measures were:
Change from baseline in FRI parameters, assessed by chest HRCT.
Change in target and/or disease specific biomarkers, in relation to genotype subgroups in blood and/or clinical endpoints over time compared to baseline

### 1.2. Study Interventions

The study provided for 2 types of interventions:
Drug: Compound 1—100 mg administered orally once daily as 2 capsules of 50 mg with or without food
Drug: Compound 1 placebo—administered orally once daily as 2 capsules with or without food

### 1.3. Study Arms

Study participants were randomized over 2 study arms:
1. Experimental: Compound 1: Compound 1 100 mg, for 26 weeks
2. Placebo Comparator: Placebo: Compound 1 placebo for 26 weeks

### 1.4. Eligibility Criteria

To be eligible, the subjects met the following criteria:
Males or females of non-child-bearing potential, aged ≥40 years.
A diagnosis of IPF within 5 years prior to the screening visit as per American Thoracic Society (ATS)/European Respiratory Society (ERS)/Japanese Respiratory Society (JRS)/Latin American Thoracic Association (ALAT) guidelines.
Meeting all of the following criteria at screening and during the screening period:
FVC≥50% predicted of normal
Disease progression in the last 9 months prior to the screening period and at screening, defined as at least one prescreening FVC value and screening value with a decline of FVC (% predicted or mL), at the investigator's discretion
Diffusing capacity for the lungs for carbon monoxide (DLCO)≥30% predicted of normal (corrected for hemoglobin)
Ratio of forced expiratory volume in one second (FEV1) to FVC≥0.70
In a stable condition and suitable for study participation based on the results of a medical history, physical examination, vital signs, 12-lead ECG, and laboratory evaluation. Stable condition is based on the clinical judgment of the investigator, co-morbidities should be treated according to the local applicable guidelines. Concomitant medication for comorbidities should be stabilized from 4 weeks before screening and during the screening period (stable defined as no change of dose or regimen).
Estimated minimum life expectancy of 12 months for non-IPF related disease in the opinion of the investigator.
Male subjects with female partners of child bearing potential are willing to comply with the contraceptive methods described in the respective protocol prior to the first dose of the IMP, during the clinical study, and for at least 12 weeks after the last dose of the IMP.
Able to walk at least 150 meters during the 6MWT at screening; without having a contraindication to perform the 6MWT.
Able to understand the importance of adherence, and willing to comply to study treatment, study procedures and requirements as per study protocol, including the concomitant medication restrictions.

Key exclusion criteria: subjects meeting one or more of the following criteria were not selected for this study:
Known hypersensitivity to any of the IMP ingredients or a history of a significant allergic reaction to any drug as determined by the investigator (e.g. anaphylaxis requiring hospitalization).
History of or a current immunosuppressive condition (e.g. human immunodeficiency virus [HIV] infection, congenital, acquired, medication induced).
Positive serology for hepatitis B (surface antigen and core antibody) or C (antibody), or any history of hepatitis from any cause with the exception of hepatitis A.
History of malignancy within the past 5 years (except for carcinoma in situ of the uterine cervix, basal cell carcinoma of the skin that has been treated with no evidence of recurrence, and prostate cancer medically managed through active surveillance or watchful waiting, and squamous cell carcinoma of the skin if fully resected).
Acute IPF exacerbation within 3 months prior to screening and during the screening period.
Lower respiratory tract infection requiring antibiotics within 4 weeks prior to screening and/or during the screening period.
Interstitial lung disease associated with known primary diseases (e.g. sarcoidosis, amyloidosis), exposures (e.g. radiation, silica, asbestos, coal dust), and drugs (e.g. amiodarone).
History of lung volume reduction surgery or lung transplant.
Unstable cardiovascular, pulmonary (other than IPF) or other disease within 6 months prior to screening or during the screening period (e.g. coronary heart disease, heart failure, stroke).
Subject participating in a drug, device or biologic investigational research study, concurrently with the current study, or within 5-half-lives of the agent (or within 8 weeks when half-life is unknown) prior to screening, or prior participation in an investigational drug antibody study within 6 months prior to screening.

Adverse events: monitoring of adverse events (AEs), serious adverse events (SAEs) and treatment-emergent adverse events (TEAEs), was conducted from initiation of the clinical trial. Assessments were made to ensure appropriate collection of safety data and to assess any perceived safety concerns.

The baseline disease characteristics for the patient population, including their distribution in the respective strata, is given in Table 1 below.

TABLE 1

| | GLPG1205, | | All |
|---|---|---|---|
| Parameter | 100 mg | Placebo | Subjects |
| Baseline background SOC, n (%) | (N = 45) | (N = 23) | (N = 68) |
| Currently taking nintedanib | 17 (37.8) | 7 (30.4) | 24 (35.3) |
| Currently taking pirfenidone | 11 (24.4) | 8 (34.8) | 19 (27.9) |
| Never took pirfenidone/nintedanib | 16 (35.6) | 8 (34.8) | 24 (35.3) |
| Stopped taking pirfenidone/ | 1 (2.2) | 0 | 1 (1.5) |
| nintedanib | | | |

The incidence of treatment-emergent adverse events (TEAEs), including those that required discontinuation of treatment, are recorded in Table 2 below.

TABLE 2

| | Background therapy | | | Summary |
|---|---|---|---|---|
| | None | Pirfenidone | Nintedanib | All |
| Subjects with, n (%) | (N = 25) | (N = 19) | (N = 24) | (N = 68) |
| Serious TEAE | 2 (8.0) | 0 | 8 (33.3) | 10 (14.7) |
| GLPG1205 100 mg | 2 | 0 | 7 | 9 |
| Placebo | 0 | 0 | 1 | 1 |
| TEAE leading to | 2 (8.0) | 2 (10.5) | 6 (25.0) | 10 (14.7) |
| treatment | | | | |
| discontinuation | | | | |
| (LD) | | | | |
| GLPG1205 100 mg | 2 | 2 | 6 | 10 |
| Placebo | 0 | 0 | 0 | 0 |

### Avoiding or Discontinuing Administration of Nintedanib to Avoid Adverse Events with Compound 1

In some aspects, the invention provides a method of administering compound 1 therapy to a patient in need of compound 1 therapy (e.g. a patient with IPF), involving administering to the patient a therapeutically effective amount of compound 1, and avoiding use or administration of nintedanib.

In other aspects, the invention provides a method of administering compound 1 therapy to a patient in need of compound 1 therapy, comprising discontinuing administration of nintedanib to avoid an adverse event, in particular a treatment-emergent adverse event (TEAE), and administering a therapeutically effective amount of compound 1.

In one example, in a method of administering a therapeutically effective amount of compound 1 to a patient with IPF, the invention provides an improvement in compound 1 therapy that comprises avoiding or discontinuing administration of nintedanib and administering a therapeutically effective amount of compound 1.

In some embodiments, nintedanib is discontinued concurrent with starting administration of compound 1.

In other embodiments, nintedanib is discontinued at least 12 hours to 1 week prior to or after starting compound 1 therapy. This time period, for example, can permit adequate time for tapering and withdrawal without adverse effects.

In embodiments in which nintedanib is discontinued to avoid an adverse drug interaction or to avoid an adverse event, in particular a treatment-emergent adverse event (TEAE), nintedanib preferably is discontinued within at least 3 days prior to starting compound 1 therapy. In various embodiments, nintedanib is discontinued within at least 4 days, or at least 5 days, or at least 6 days, or at least 7 days (or one week), or at least 8 days, or at least 9 days, or at least 10 days, or at least 11 days, or at least 12 days, or at least 13 days, or at least 14 days (or two weeks), or at least 15 days, or at least 16 days, or at least 17 days, or at least 18 days, or at least 19 days, or at least 20 days, or at least 21 days (or three weeks), or at least 22 days, or at least 23 days, or at least 24 days, or at least 25 days, or at least 26 days, or at least 27 days, or at least 28 days (or four weeks), or at least 29 days, or at least 30 days, or at least one month, prior to starting compound 1 therapy. In some embodiments, nintedanib is discontinued no earlier than one month, 3 weeks, 2 weeks or 1 week before starting compound 1 therapy. Preferably, sufficient time is allowed for tapering and/or withdrawal of nintedanib.

In embodiments where nintedanib cannot be or is not discontinued prior compound 1 therapy, nintedanib is preferably discontinued within at least 3 days after starting compound 1 therapy.

The patient preferably avoids use of nintedanib to allow sufficient time to avoid adverse events following starting compound 1 therapy.

### Selecting an Alternative Drug or Therapy to Administer Concurrently with Compound 1 Therapy

In some aspects, the invention provides a method of administering compound 1 therapy to a patient in need of compound 1 therapy and in need of therapy with nintedanib comprising administering a therapeutically effective amount of compound 1 to the patient, and administering an alternative therapy that is not nintedanib. In one embodiment according to these aspects, the method comprises concomitant use or co-administration of pirfenidone. In another embodiment according to these aspects, the method comprises discontinuing treatment with nintedanib and commencing treatment with pirfenidone.

### Improving Administration of Compound 1 Therapy by Advising or Cautioning Patient

The administration of a therapeutically effective amount of compound 1 to a patient in need of compound 1 therapy can be improved. In some embodiments, the patient is advised that co-administration of compound 1 with nintedanib can alter the therapeutic effect or adverse reaction profile of compound 1 and/or nintedanib.

In some embodiments, the patient, if receiving nintedanib therapy, is advised that co-administration of compound 1 with nintedanib can alter the therapeutic effect or adverse reaction profile of compound 1 and/or nintedanib and that nintedanib therapy should be discontinued at or prior to commencing compound 1 therapy.

### Packages, Kits, Methods of Packaging, and Methods of Delivering

In another aspect, a package or kit is provided comprising compound 1, optionally in a container, and, a package insert, package label, instructions or other labelling including information, recommendation or instruction regarding the avoidance or discontinuation or contraindication of concurrent use of compound 1 and nintedanib in general, as described in different aspects and embodiments herein. E.g. such as a package insert, package label, instructions or other labelling may include any one or more of the following information, recommendation or instruction:
Informing or advising the patient that concurrent use of nintedanib should be avoided;
Informing or advising the patient that concurrent use of nintedanib is contraindicated;
Informing or advising the patient that concurrent use of nintedanib should be discontinued, e.g. for at least 12 hours, 1 day, 2 days, 3 days, 4 days, 5 days, 6 days, 1 week, at least two weeks, at least 3 weeks or at least 4 weeks prior to compound 1 therapy;
Informing or advising the patient that concurrent use of nintedanib can alter the therapeutic effect of nintedanib or of compound 1, e.g. decreases the therapeutic effect of compound 1 or nintedanib, and/or lead to adverse events, in which case the instruction may further state that therefore concurrent use is contraindicated;
Instructing the patient to discontinue concurrent use of nintedanib;
Instructing the patient in need of compound 1 therapy to not use or administer concurrently nintedanib;
Contra-indicating the concurrent use or administration of nintedanib.

### Embodiments of the Invention

Further examples of embodiments of the invention include those given directly below:

1. Compound 1 for use in treating a patient in need of compound 1 therapy, characterized in that the treating comprises avoiding or contraindicating or discontinuing concomitant use or co-administration of nintedanib.
2. The use of compound 1 in the manufacture of a medicament for treating a patient in need of therapy using compound 1, characterized in that the treating comprises avoiding or contraindicating or discontinuing concomitant use or co-administration of nintedanib.
3. A method of administering treatment using compound 1 to a patient in need of therapy using compound 1 comprising administering the patient a therapeutically effective amount of compound 1, and, avoiding or contraindicating or discontinuing concomitant use or co-administration of nintedanib.
4. Compound 1 for use, use of compound 1 or method according to any of the preceding embodiments wherein the patient in need of therapy using compound 1 is currently undergoing treatment with nintedanib.
5. Compound 1 for use, use of compound 1 or method according to any of the preceding embodiments wherein the use or method comprises the step of discontinuing the use of or treatment with nintedanib prior to or at the same time as the step of starting compound 1 therapy.
6. Compound 1 for use, use of compound 1 or method according to embodiment 5 wherein the use of or treatment with nintedanib is discontinued at least 12 hours, preferably 24 hours, prior to commencing compound 1 therapy.
7. Compound 1 for use, use of compound 1 or method according to any of the preceding embodiments wherein the use or method further comprises concomitant use or co-administration of pirfenidone.
8. Compound 1 for use, use of compound 1 or method according to any of the preceding embodiments wherein the use or method comprises discontinuing treatment with nintedanib and commencing treatment with pirfenidone.
9. Compound 1 for use, use of compound 1 or method according to any of the preceding embodiments wherein the patient in need of compound 1 therapy has pulmonary fibrosis, such as idiopathic pulmonary fibrosis (IPF) or progressive fibrosing interstitial lung disease (PF-ILD).
Compound 1 for use, use of compound 1 or method according to any of the preceding embodiments wherein compound 1 is administered at a total daily dosage of at least 25 to 300 mg.
11. Compound 1 for use, use of compound 1 or method according to any of the preceding embodiments wherein compound 1 is administered at a total daily dosage of at least 25 to 200 mg.
12. Compound 1 for use, use of compound 1 or method according to any of the preceding embodiments wherein compound 1 is administered at a total daily dosage of at least 25 to 100 mg.
13. Compound 1 for use, use of compound 1 or method according to any of the preceding embodiments wherein each dose of compound 1 administered is 25, 50 or 100 mg.
14. Compound 1 for use, use of compound 1 or method according to any of the preceding embodiments wherein each dose of compound 1 administered is 25 or 50 mg.
A package or kit comprising (i) compound 1, and (ii) a package insert, package label, instructions or other labelling comprising instructions to avoid or discontinue or contraindication of concomitant use or co-administration of nintedanib.
16. A package or kit according to embodiment 15 further comprising one or more of the features according to embodiments 1 to 14.

### Final Remarks

It will be appreciated by those skilled in the art that the foregoing descriptions are exemplary and explanatory in nature, and intended to illustrate the invention and its preferred embodiments. Through routine experimentation, an artisan will recognize apparent modifications and variations that may be made without departing from the spirit of the invention. All such modifications coming within the scope of the appended claims are intended to be included therein. Thus, the invention is intended to be defined not by the above description, but by the following claims and their equivalents.

All publications, including but not limited to patents and patent applications, cited in this specification are herein incorporated by reference as if each individual publication are specifically and individually indicated to be incorporated by reference herein as though fully set forth.

It should be understood that factors such as the differential cell penetration capacity of the compound can contribute to discrepancies between the activity of the compound in the in vitro biochemical and cellular assays.

At least some of the chemical names of compound of the invention as given and set forth in this application, may have been generated on an automated basis by use of a commercially available chemical naming software program, and have not been independently verified. Representative programs performing this function include the Lexichem naming tool sold by Open Eye Software, Inc. and the Autonom Software tool sold by MDL, Inc. In the instance where the indicated chemical name and the depicted structure differ, the depicted structure will control.

### REFERENCES

Ashcroft, T., Simpson, J. M., Timbrell, V., 1988. Simple method of estimating severity of pulmonary fibrosis on a numerical scale. J. Clin. Pathol. 41, 467-470.
Bickelhaupt, S., Erbel, C., Timke, C., Wirkner, U., Dadrich, M., Flechsig, P., Tietz, A., Pföhler, J., Gross, W., Peschke, P., Hoeltgen, L., Katus, H. A., Grone, H.-J., Nicolay, N. H., Saffrich, R., Debus, J., Sternlicht, M. D., Seeley, T. W., Lipson, K. E., Huber, P. E., 2017. Effects of CTGF Blockade on Attenuation and Reversal of Radiation-Induced Pulmonary Fibrosis. JNCI J. Natl. Cancer Inst. 109. https://doi.org/10.1093/jnci/djw339
Brunnemer, E., Wälscher, J., Tenenbaum, S., Hausmanns, J., Schulze, K., Seiter, M., Heussel, C. P., Warth, A., Herth, F. J. F., Kreuter, M., 2018. Real-World Experience with Nintedanib in Patients with Idiopathic Pulmonary Fibrosis. Respiration 95, 301-309. https://doi.org/10.1159/000485933
Brunt, E. M., Kleiner, D. E., Wilson, L. A., Belt, P., Neuschwander-Tetri, B. A., 2011. The NAS and The Histopathologic Diagnosis in NAFLD: Distinct Clinicopathologic Meanings. Hepatol. Baltim. Md 53, 810-820. https://doi.org/10.1002/hep.24127
Day, C. P., James, O. F. W., 1998. Hepatic steatosis: Innocent bystander or guilty party? Hepatology 27, 1463-1466. https://doi.org/10.1002/hep.510270601
Devos, F. C., Maaske, A., Robichaud, A., Pollaris, L., Seys, S., Lopez, C. A., Verbeken, E., Tenbusch, M., Lories, R., Nemery, B., Hoet, P. H., Vanoirbeek, J. A., 2017. Forced expiration measurements in mouse models of obstructive and restrictive lung diseases. Respir. Res. 18, 123. https://doi.org/10.1186/s12931-017-0610-1
Fujii, T., Fuchs, B. C., Yamada, S., Lauwers, G. Y., Kulu, Y., Goodwin, J. M., Lanuti, M., Tanabe, K. K., 2010. Mouse model of carbon tetrachloride induced liver fibrosis: Histopathological changes and expression of CD133 and epidermal growth factor. BMC Gastroenterol. 10, 79. https://doi.org/10.1186/1471-230X-10-79
Inghilleri, S., Morbini, P., Oggionni, T., Barni, S., Fenoglio, C., 2006. In situ assessment of oxidant and nitrogenic stress in bleomycin pulmonary fibrosis. Histochem. Cell Biol. 125, 661-669. https://doi.org/10.1007/s00418-005-0116-7
Kleiner, D. E., Brunt, E. M., Van Natta, M., Behling, C., Contos, M. J., Cummings, O. W., Ferrell, L. D., Liu, Y.-C., Torbenson, M. S., Unalp-Arida, A., Yeh, M., McCullough, A. J., Sanyal, A. J., Nonalcoholic Steatohepatitis Clinical Research Network, 2005. Design and validation of a histological scoring system for nonalcoholic fatty liver disease. Hepatol. Baltim. Md 41, 1313-1321. https://doi.org/10.1002/hep.20701
Lancaster, L. H., Andrade, J. A. de, Zibrak, J. D., Padilla, M. L., Albera, C., Nathan, S. D., Wijsenbeek, M. S., Stauffer, J. L., Kirchgaessler, K.-U., Costabel, U., 2017. Pirfenidone safety and adverse event management in idiopathic pulmonary fibrosis. Eur. Respir. Rev. 26, 170057. https://doi.org/10.1183/16000617.0057-2017
Marra, F., DeFranco, R., Grappone, C., Milani, S., Pastacaldi, S., Pinzani, M., Romanelli, R. G., Laffi, G., Gentilini, P., 1998. Increased expression of monocyte chemotactic protein-1 during active hepatic fibrogenesis: correlation with monocyte infiltration. Am. J. Pathol. 152, 423-430.
Matsuse, T., Teramoto, S., Katayama, H., Sudo, E., Ekimoto, H., Mitsuhashi, H., Uejima, Y., Fukuchi, Y., Ouchi, Y., 1999. ICAM-1 mediates lung leukocyte recruitment but not pulmonary fibrosis in a murine model of bleomycin-induced lung injury. Eur. Respir. J. 13, 71-77.
Matsuzawa, Y., Kawashima, T., Kuwabara, R., Hayakawa, S., Irie, T., Yoshida, T., Rikitake, H., Wakabayashi, T., Okada, N., Kawashima, K., Suzuki, Y., Shirai, K., 2015. Change in serum marker of oxidative stress in the progression of idiopathic pulmonary fibrosis. Pulm. Pharmacol. Ther. 32, 1-6. https://doi.org/10.1016/j.pupt.2015.03.005
Miyamoto, J., Hasegawa, S., Kasubuchi, M., Ichimura, A., Nakajima, A., Kimura, I., 2016. Nutritional Signaling via Free Fatty Acid Receptors. Int. J. Mol. Sci. 17. https://doi.org/10.3390/ijms17040450
Nanthakumar, C. B., Hatley, R. J. D., Lemma, S., Gauldie, J., Marshall, R. P., Macdonald, S. J. F., 2015. Dissecting fibrosis: therapeutic insights from the small-molecule toolbox. Nat. Rev. Drug Discov. 14, 693-720. https://doi.org/10.1038/nrd4592
Neuschwander-Tetri, B. A., Caldwell, S. H., 2003. Nonalcoholic steatohepatitis. Summary of an AASLD Single Topic Conference. Hepatology 37, 1202-1219. https://doi.org/10.1053/jhep.2003.50193 Richeldi, L., du Bois, R. M., Raghu, G., Azuma, A., Brown, K. K., Costabel, U., Cottin, V., Flaherty, K. R., Hansell, D. M., Inoue, Y., Kim, D. S., Kolb, M., Nicholson, A. G., Noble, P. W., Selman, M., Taniguchi, H., Brun, M., Le Maulf, F., Girard, M., Stowasser, S., Schlenker-Herceg, R., Disse, B., Collard, H. R., INPULSIS Trial Investigators, 2014. Efficacy and safety of nintedanib in idiopathic pulmonary fibrosis. N. Engl. J. Med. 370, 2071-2082. https://doi.org/10.1056/NEJMoa1402584
Richter, K., Kietzmann, T, 2016. Reactive oxygen species and fibrosis: further evidence of a significant liaison. Cell Tissue Res. 365, 591-605. https://doi.org/10.1007/s00441-016-2445-3
Santhekadur, P. K., Kumar, D. P., Sanyal, A. J., 2017. Preclinical Models of Nonalcoholic Fatty Liver Disease. J. Hepatol. https://doi.org/10.1016/j jhep.2017.10.031
Suzuki, M., Takaishi, S., Nagasaki, M., Onozawa, Y., Iino, I., Maeda, H., Komai, T., Oda, T., 2013. Medium-chain Fatty Acid-sensing Receptor, GPR84, Is a Proinflammatory Receptor. J. Biol. Chem. 288, 10684-10691. https://doi.org/10.1074/jbc.M112.420042
Wang, J., Wu, X., Simonavicius, N., Tian, H., Ling, L., 2006. Medium-chain Fatty Acids as Ligands for Orphan G Protein-coupled Receptor GPR84. J. Biol. Chem. 281, 34457-34464. https://doi.org/10.1074/jbc.M608019200
Wittenberger, T., Schaller, H. C., Hellebrand, S., 2001. An expressed sequence tag (EST) data mining strategy succeeding in the discovery of new G-protein coupled receptors. J. Mol. Biol. 307, 799-813. https://doi.org/10.1006/jmbi.2001.4520
Yousefi, S., Cooper, P. R., Potter, S. L., Mueck, B., Jarai, G., 2001. Cloning and expression analysis of a novel G-protein-coupled receptor selectively expressed on granulocytes. J. Leukoc. Biol. 69, 1045-1052.
Zimmermann, H. W., Seidler, S., Nattermann, J., Gassler, N., Hellerbrand, C., Zernecke, A., Tischendorf, J. J. W., Luedde, T., Weiskirchen, R., Trautwein, C., Tacke, F., 2010. Functional Contribution of Elevated Circulating and Hepatic Non-Classical CD14+CD16+ Monocytes to Inflammation and Human Liver Fibrosis. PLOS ONE 5, e11049. https://doi.org/10.1371/journal.pone.0011049

1. Compound 1 for use in treating a patient in need of compound 1 therapy, characterized in that the treating comprises avoiding or contraindicating or discontinuing concomitant use or co-administration of nintedanib.

2. The use of compound 1 in the manufacture of a medicament for treating a patient in need of therapy using compound 1, characterized in that the treating comprises avoiding or contraindicating or discontinuing concomitant use or co-administration of nintedanib.

3. A method of administering treatment using compound 1 to a patient in need of therapy using compound 1 comprising administering the patient a therapeutically effective amount of compound 1, and, avoiding or contraindicating or discontinuing concomitant use or co-administration of nintedanib.

4. Compound 1 for use, use of compound 1 or method according to any of the preceding claims wherein the patient in need of therapy using compound 1 is currently undergoing treatment with nintedanib.

5. Compound 1 for use, use of compound 1 or method according to any of the preceding claims wherein the use or method comprises the step of discontinuing the use of or treatment with nintedanib prior to or at the same time as the step of starting compound 1 therapy.

6. Compound 1 for use, use of compound 1 or method according to claim 5 wherein the use of or treatment with nintedanib is discontinued at least 12 hours, preferably 24 hours, prior to commencing compound 1 therapy.

7. Compound 1 for use, use of compound 1 or method according to any of the preceding claims wherein the use or method further comprises concomitant use or co-administration of pirfenidone.

8. Compound 1 for use, use of compound 1 or method according to any of the preceding claims wherein the use or method comprises discontinuing treatment with nintedanib and commencing treatment with pirfenidone.

9. Compound 1 for use, use of compound 1 or method according to any of the preceding claims wherein the patient in need of compound 1 therapy has pulmonary fibrosis, such as idiopathic pulmonary fibrosis (IPF) or progressive fibrosing interstitial lung disease (PF-ILD).

10. Compound 1 for use, use of compound 1 or method according to any of the preceding claims wherein compound 1 is administered at a total daily dosage of at least 25 to 300 mg.

11. Compound 1 for use, use of compound 1 or method according to any of the preceding claims wherein each dose of compound 1 administered is 25, 50 or 100 mg.

12. A package or kit comprising (i) compound 1, and (ii) a package insert, package label, instructions or other labelling comprising instructions to avoid or discontinue or contraindication of concomitant use or co-administration of nintedanib.

13. A package or kit according to claim 12 further comprising one or more of the features according to claims 1 to 11.
